(19) United States
(12) Patent Application Publication
(10) Pub. No.: US 2015/0273232 A1
(43) Pub. Date: Oct. 1, 2015

(54) Athletic Performance Enhancing Wear and Method

(71) Applicant: Miguel Angel Fernandez, Weston, FL (US)

(72) Inventor: Miguel Angel Fernandez, Weston, FL (US)

(21) Appl. No.: 14/225,083

(22) Filed: Mar. 25, 2014

(51) Int. Cl. A61N 2/00; A61H 39/00

(52) U.S. Cl. CPC A61N 2/004; A61H 39/00; A61H 2201/165

(57) ABSTRACT

An article of clothing comprising of magnets and a method to stimulate specific combination of acupuncture points on the human body related to athletic performance.

### TECHNICAL FIELD

The present invention is in the technical field of athletics. More particularly, the present invention is in the technical field of athletic performance devices. More particularly, the present invention is in the technical field of athletic performance enhancing clothing.

### BACKGROUND AND PRIOR ART

Acupuncture has been a key component of traditional Chinese medicine for thousands of years and more recently in the western world it has been accepted as a respected form of therapy. Acupuncture points are often treated with needles or acupressure massage. More recently the application of magnets to these points has shown to be just as effective as needles or other forms of stimulation as used in magnetic therapy. Magnetic Therapy is an alternative medical therapy in which the placement of magnets on the skin is on areas of the body is used to treat pain. Magnetic therapy has been used broadly around the world and many companies have many product related to this type of treatment.

Other patents have been granted that deal with magnetic therapies, acupuncture methods and devices embedded in clothing. For example U.S. Pat. No. 6,258,020 teaches a variety of specialized wearable merchandise provided with the magnetic structure that is worn by humans for magnetic exposure therapeutic reasons. Other prior art involves clothing containing magnets for the topical placement of magnets in strategic locations for the treatment of ailments on patients and other therapeutic reason related to pain relieve. However these prior inventions are primarily therapeutic, by definition related to medical treatment of a disease or condition that utilize magnetic and/or acupuncture method or device for treatment. Some products with prior granted patents related to wearable items are intended for the use of health a practitioner or therapist on patients, or a health practitioner would be necessary to determine the specific number, combination and location of acupuncture points to be used by the patient/user when utilizing the device. These inventions and existing products on the market, as well as those which are ready to be used by the “end user” without the aid of a therapist or health practitioner are built specifically for therapeutic reasons, with the acupuncture or acupressure pattern structures designed exclusively for treatment of health problems or pain relieve.

### SUMMARY

A need is seen to exist for a larger variety of specialized merchandise provided with the magnetic structure design to allow the end user to help achieve an improved athletic performance.

The present invention is an article of clothing incorporating magnets to stimulate specific combination of acupuncture points on the human body to obtain the desired effect; more specifically, athletic performance improvement.

An article of clothing comprising of the magnetic structure based on a combination of acupuncture points that is worn by a person for the purpose of enhancing the athletic performance, prior, during or after an athletic activity of the person. In particular, a need is seen to exist for merchandise in the form of torso worn garments provided with the magnetic structure based on various combinations of acupuncture points on the human torso for effecting stimulation of these points by magnetic exposure when worn by a user, helping respiration, muscle, blood flow and other areas which help improve the physical performance of a the user.

### DESCRIPTION

A particular object of the present invention is to provide merchandise in the form of torso worn garments comprising of the magnetic structure that corresponds to selected acupuncture points on the human torso that can be related to physical performance for effecting magnetic exposure when worn by a human user.

An important object of the present invention is to provide a method to enhance the athletic performance on humans. The objective is achieved by selecting a various specific combinations of acupuncture points that are associated with the areas of the human body that are affected by specific athletic activities and/or that could influence the capability of the body when performing an athletic activity.

Another particular object of the present invention is to provide a variety of merchandise in the form of wearable gear provided with the magnetic structure that corresponds to selected acupuncture points on the human body that can be related to physical performance for effecting magnetic exposure when worn by a human user.

### BRIEF DESCRIPTION OF THE DRAWINGS

FIG. 1 is a front view of a torso worn garment of the present invention; showing a short sleeve and a long sleeve option;

FIG. 2 is a back view of a torso worn garment of the present invention; showing a short sleeve and a long sleeve option.

### DETAILED DESCRIPTION OF THE INVENTION:

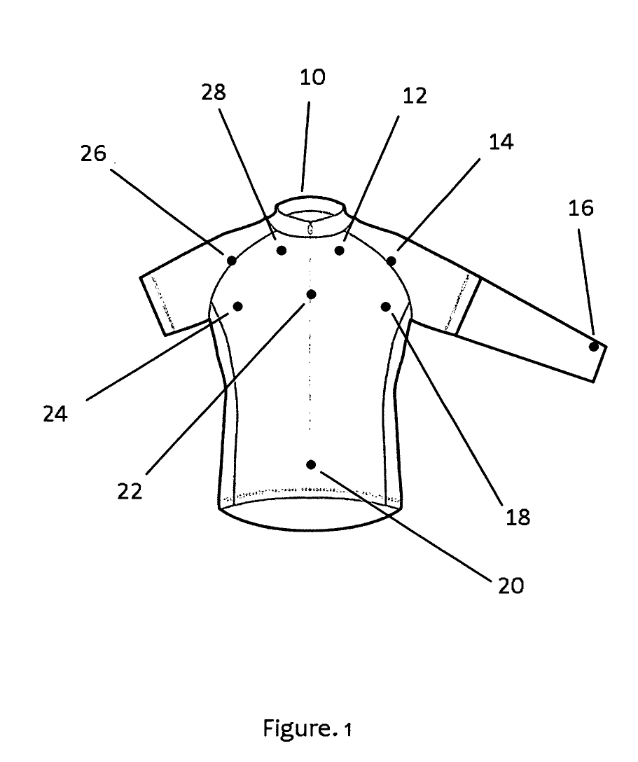

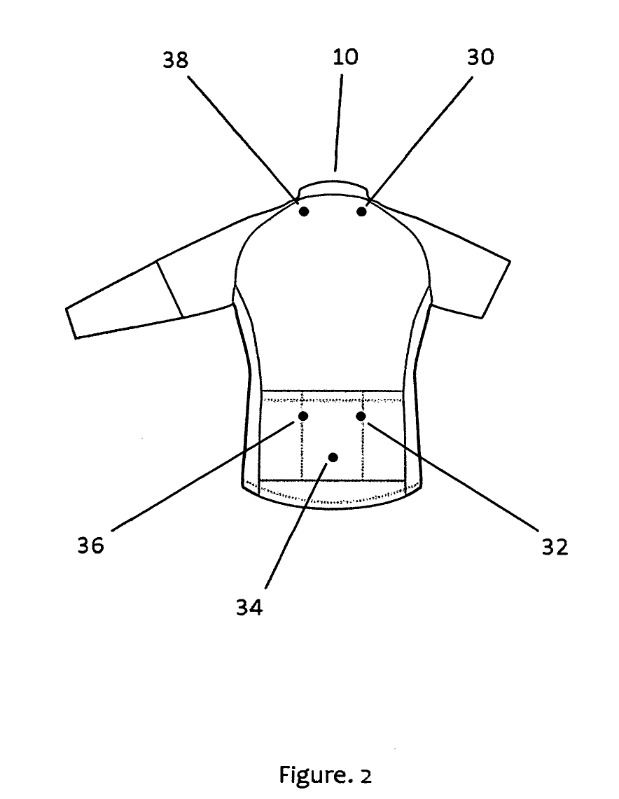

Referring now to the invention in more detail, in FIG. 1 there is shown a front view of a torso worn garment 10 having the magnetic structure corresponding to acupuncture points: Left KD27 12, Left LU1 14, Left TH5 16, Left CS1 18, CV6 20, CV17 22, Right CS1 24, Right LU1 26 and Right KD27 28. FIG. 2 shows a back view of a torso worn garment 10 having the magnetic structure corresponding to acupuncture points: Right UB36 30, Right UB46 32, GV4 34, Left UB46 36 and Left UB36 38.

In more detail, still referring to the invention of FIG. 1, the front view of a torso worn garment 10 shows the location where magnets are placed corresponding with the combination of acupuncture points: Left KD27 12, Left LU1 14, Left TH5 16, Left CS1 18, CV6 20, Right CV17 22, Right CS1 24, Right LU1 26 and Right KD27 28. Magnets are used as a mean for stimulation of said points on the human body by magnetic exposure when worn by a user. Referring to the invention in FIG. 2, the back view of a torso worn garment 10 shows the location where magnets are placed corresponding with the combination of acupuncture points: Right UB36 30, Right UB46 32, GV4 34, Left UB46 36 and Left UB36 38. Magnets are used as a mean for stimulation of said points on the human body by magnetic exposure when worn by a user.

In further detail, still referring to the invention of FIG. 1 and FIG. 2, the magnets are placed on each acupuncture point Left KD27 12, Left LU1 14, Left TH5 16, Left CS1 18, CV6 20, Right CV17 22, Right CS1 24, Right LU1 26, Right KD27 28, Right UB36 30, Right UB46 32, GV4 34, Left UB46 36 and Left UB36 38, in such a way that each acupuncture point 12, 14, 16, 18, 20, 22, 24, 26, 28, 30, 32, 34, 36 and 38 is in contact with a magnet and sufficiently enough area around each acupuncture point 12, 14, 16, 18, 20, 22, 24, 26, 28, 30, 34, 36 and 38, such as about ½ to 1½ inches in diameter is in contact with a magnet.

The construction details of the invention as shown in FIG. 1 are that the torso worn garment 10 may be made of moisture-wicking fabric, compression type fabric or of any other type of fabric such as spandex, cotton, and the like. Further, the various components of the torso worn garment 10 can be made of different materials.

The magnets may be flat disk shaped, or of any other shape. Further, one magnet or a larger number of magnets may be used to cover each acupuncture point 12, 14, 16, 18, 20, 22, 24, 26, 28, 30, 34, 36 and 38 areas.

The advantages of the present invention include, without limitation that it is product ready to be used by the end user without the aid of a therapist or health practitioner. It is designed to help achieve an improved athletic performance rather than for treatment of health problems or pain relieve. Further, it allows the end user to receive stimulation prior, during or after an athletic activity. Further, it is easy to use since it is worn by the end user in the same manner as a regular shirt.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A method to enhance the athletic performance on humans by stimulating specific combinations of selected acupuncture points that are associated with the areas of the human body that are related with specific athletic activities.

2. The method to enhance the athletic performance on humans according to claim 1, wherein the said specific combination of selected acupuncture points are stimulated by the use of magnets.

3. The method to enhance the athletic performance on humans according to claim 1, wherein the said specific combination of selected acupuncture points are stimulated by the use of magnets according to claim 2, wherein the said selected combination of acupuncture points consist of points Left KD27, Left LU1, Left TH5, Left CS1, CV6, Right CV17, Right CS1, Right LU1, Right KD27, Right UB36, Right UB46, GV4, Left UB46 and Left UB36.

4. An article of clothing comprising of the magnetic structure that corresponds to selected combination of acupuncture points on the human body associated to physical performance for effecting magnetic exposure when worn by a human user and thereby stimulating said points to obtain athletic performance improvement.

5. The article of clothing according to claim 4, more specifically, a torso worn garment, wherein the said magnetic structure corresponding to selected acupuncture points consist of points: Left KD27, Left LU1, Left TH5, Left CS1, CV6, Right CV17, Right CS1, Right LU1, Right KD27, Right UB36, Right UB46, GV4, Left UB46 and Left UB36.
